(19) United States
(12) Patent Application Publication
(10) Pub. No.: US 2016/0230206 A1
(43) Pub. Date: Aug. 11, 2016

(54) SMART (SUBSURFACE MICROBIAL ACTIVITY IN REAL TIME) TECHNOLOGY FOR REAL-TIME MONITORING OF SUBSURFACE MICROBIAL METABOLISM

(71) Applicant: UNIVERSITY OF MASSACHUSETTS, Boston, MA (US)

(72) Inventors: Derek R. Lovley, Amherst, MA (US); Kelly Nevin, Amherst, MA (US)

(21) Appl. No.: 15/131,421

(22) Filed: Apr. 18, 2016

Related U.S. Application Data
(63) Continuation of application No. PCT/US14/60635, filed on Oct. 15, 2014.
(60) Provisional application No. 61/892,158, filed on Oct. 17, 2013.

(51) Int. Cl. C12Q 1/02

(52) U.S. Cl. CPC C12Q 1/02

(57) ABSTRACT

A sensor that measures microbial activity as a surrogate value for the biologically active content of soil, aquatic sediments, or groundwater. An anode, such as a graphite anode that can support a biofilm, is connected by way of a resistor to a cathode. The anode is in contact with either soil, sediment, or immersed in the groundwater of a subsurface monitoring well. The biofilm generates electrons as a consequence of chemical interactions with materials such as acetate dissolved in the soil or sediment waters or groundwater. The cathode is located in soil or water adjacent to the ground, which can be aerobic, so that a reaction that consumes electrons occurs at the cathode. The current flowing through the resistor is a measure of the biological activity at the anode, which correlates with the flux of fuel such as acetate to the anode.

### CROSS-REFERENCE TO RELATED APPLICATIONS

This application is a continuation of co-pending International Patent Application No. PCT/US14/60635 filed Oct. 15, 2014, which application claims priority to and the benefit of then co-pending U.S. provisional patent application Ser. No. 61/892,158, filed Oct. 17, 2013, each of which applications is incorporated herein by reference in its entirety.

### STATEMENT REGARDING FEDERALLY FUNDED RESEARCH OR DEVELOPMENT

This invention was made with government support under Grant DE-SC0006790 awarded by the Department of Energy. The government has certain rights in the invention.

### THE NAMES OF THE PARTIES TO A JOINT RESEARCH AGREEMENT

A joint research agreement between University of Massachusetts (Derek Lovley, PI) and Lawrence Berkeley National Laboratory (Kenneth H. Williams, performing field tests using the sensors provided by Lovley) has been entered into as part of the Department of Energy contract identified above.

### FIELD OF THE INVENTION

The invention relates to microbial activity sensors in general and particularly to a microbial activity sensor that operates in situ.

### BACKGROUND OF THE INVENTION

Anaerobic microbial processes play an important role in the biogeochemistry of submerged soils and aquatic sediments, as well as in deeper subsurface environments (see Lovley D R, Chapelle F H. 1995. Deep subsurface microbial processes. Rev. Geophsy. 33:365-381). Which anaerobic process predominates within a given environment can be simply determined from measurements of steady-state H₂ concentrations (see Lovley D R, Goodwin S. 1988. Hydrogen concentrations as an indicator of the predominant terminal electron accepting reactions in aquatic sediments. Geochim. Cosmochim. Acta. 52:2993-3003). However, assessing the rates of anaerobic processes has proven to be much more difficult.

Most strategies for estimating rates of microbial metabolism involve incubating soil/sediment subsamples, which can dramatically change rates of microbial activity (see Chapelle F H, Lovley D R. 1990. Rates of microbial metabolism in deep coastal plain aquifers. Appl. Environ. Microbiol. 56:1865-1874) and typically require sophisticated analytical techniques for analyzing the products of microbial metabolism. The labor and expense of such measurements often negate the possibility of making detailed time series of microbial rate measurements that are required for studies on the response of microbial activity to seasonal changes or environmental disturbances, such as the introduction of contaminants.

The ability of microorganisms to produce current in response to the availability of an organic substrate that was externally provided was previously demonstrated in laboratory systems containing water (see Bond D R, Holmes D E, Tender L M, Lovley D R. 2002. Electrode-reducing microorganisms that harvest energy from marine sediments. Science 295:483-485; Bond D R, Lovley D R. 2003. Electricity production by Geobacter sulfurreducens attached to electrodes. Appl. Environ. Microbiol. 69:1548-1555; and Chaudhuri S K, Lovley D R. 2003. Electricity generation by direct oxidation of glucose in mediatorless microbial fuel cells. Nat. Biotechnol. 21:1229-1232.) or water saturated soils (see Tront J M, Fortner J D, Plotze M, Hughes J B, Puzrin A M. 2008. Microbial fuel cell biosensor for in situ assessment of microbial activity. Biosen. Bioelectron. 24:586-590.), as well as in groundwater (see Williams K N, Nevin K P, Franks A E, Englert A, Long P E, Lovley D R. 2010. Electrode-based approach for monitoring in situ microbial activity during subsurface bioremediation. Environ. Sci. Technol. 44:47-54). However, in each of these cases the electrical response was due to an organic compound that was artificially added to the system. Furthermore, with the exception of Williams et al., the current response was attributed to microorganisms added to the system, rather than relying on the natural, indigenous microorganisms.

Friedman et al. (Friedman E S, Rosenbaum M, Lee A W, Lipson D A, Land B R, Angenent L T. 2012. A cost-effective and field-ready potentiostat that poises subsurface electrodes to monitor bacterial respiration. Biosen. Bioelectron. 32:309-313) measured current in soils and suggested that the current might be related to changes in microbial activity, but also noted that this could be a chemical reaction. Furthermore, their system relied on a technically complicated poised anode that required special electronics to maintain the poise. The poised system of Friedman et al. also requires a reference electrode for poising the anode. Reference electrodes are expensive and fragile. The need for a reference electrode greatly limits the design for deployment and feasible depth resolution because of the need to house the reference electrode in addition to the anode. The fragility of reference electrodes also complicates deployment. Furthermore the need for a reference electrode reduces the time that the sensor will be functional because reference electrodes have limited stability.

Furthermore, although microbial activity may be directly linked to the concentrations of readily degradable organic substrates in artificial environments, such as wastewater digesters, or when organic substrates are added to promote groundwater bioremediation, there is not a clear link between the concentrations of readily measured substrates and microbial activity in most anaerobic soils and sediments. In fact, the pool sizes of readily degradable organic substrates such as fermentable sugars and amino acids, as well as acetate and H₂, the prime intermediates for anaerobic respiration, are uniformly low regardless of the rates of metabolism. Rates of microbial metabolism are reflected in the turnover rates of these substrate pools, not their concentrations. For example, this is clearly evident with the fermentation product H₂. The H₂-consuming microbial community rapidly adjusts to variations in the rate of H₂ production and maintains the H₂ pool at concentrations that are just high enough that H₂ oxidation is still thermodynamically favorable with the most electro-positive electron acceptor that is available for H₂ oxidation. Therefore, environments that differ in rates of H₂ production even by an order of magnitude will have approximately the same H₂ concentrations if the same terminal electron accepting process predominates. The difference in the H₂ production rates will be reflected in the size of the H₂-consuming microbial community, the environment with a 10-fold higher rate of H₂ production will have a correspondingly higher biomass of H₂-consuming microorganisms coupled with a correspondingly higher rate of the reduction of terminal electron acceptors. Similar considerations apply to other substrates.

Therefore, when an electrode is provided as an alternative electron acceptor, the amount of current generated can also be expected to be related to the turnover rate of electron donors that can contribute to current production. Acetate is typically the most important intermediary in carbon and electron flow in anaerobic sediments and acetate-oxidizing microorganisms typically predominate on current-harvesting electrodes inserted in anaerobic soils and sediments. (See for example Lovley D R. 2006. Bug juice: harvesting electricity with microorganisms. Nature Rev. Microbiol. 4:497-508; and Lovley D R, Ueki T, Zhang T, Malvankar N S, Shrestha P M, Flanagan K, Aklujkar M, Butler J E, Giloteaux L, Rotaru A-E, Holmes D E, Franks A E, Orellana R, Risso C, Nevin K P. 2011. Geobacter: the microbe electric's physiology, ecology, and practical applications. Adv. Microb. Physiol. 59:1-100.) The rate that all of these potential electron donors are produced from complex organic material near an anode inserted in anaerobic soils and sediments should be reflected in the amount of current production. If so, there should be a direct correlation between rates of acetate turnover and current production in sediments with different rates of microbial metabolism because changes in the rate of organic matter metabolism will be accompanied by a corresponding change in the acetate turnover rate. Other organic electron donors, as well as H₂, and inorganic products of microbial metabolism may also make contributions to current production in a similar manner which is directly related to the overall rates of microbial metabolism.

There is a need for systems and methods for monitoring microbial activity that operate in situ.

### SUMMARY OF THE INVENTION

According to one aspect, the invention features a microbial activity sensor. The microbial activity sensor comprises an anode electrode configured to support a biofilm on a surface thereof, and configured to be imbedded in the ground, in sediment, or immersed in groundwater, said anode electrode having an anode electrode terminal; a cathode electrode in electrical contact with the ground, sediment, or groundwater, said cathode electrode having a cathode electrode terminal; an electrical resistance connected between said anode electrode terminal and said cathode electrode terminal; and an electrical current monitor in electrical communication with said electrical resistance, said electrical current monitor configured to measure an electrical current passing through said electrical resistance, said electrical current monitor configured to record said a value representing measured electrical current at selected times, said electrical current monitor configured to report said recorded values representing measured electrical current and said selected times in response to an interrogation command.

In one embodiment, the microbial activity sensor further comprises a communication device configured to receive said interrogation command, and configured to provide said recorded values representing measured electrical current and said selected times in response to said interrogation command.

According to another aspect, the invention relates to a microbial activity monitoring method. The method comprises the steps of providing an microbial activity sensor, operating said microbial activity sensor to generate values representing measured electrical current at selected times; recording said values representing measured electrical current and respective selected times; interrogating said microbial activity sensor to recover said values representing measured electrical current and respective selected times; analyzing said values representing measured electrical current and respective selected times to produce a result representing a level of microbial activity; and performing at least one of recording said result, transmitting said result to a data handling system, or to displaying said result to a user. The microbial activity sensor comprises an anode electrode configured to support a biofilm on a surface thereof, and configured to be imbedded in the ground, in sediment, or immersed in groundwater, said anode electrode having an anode electrode terminal; a cathode electrode configured to be in electrical contact with the ground, sediment, or groundwater, said cathode electrode having a cathode electrode terminal; an electrical resistance connected between said anode electrode terminal and said cathode electrode terminal; and an electrical current monitor in electrical communication with said electrical resistance, said electrical current monitor configured to measure an electrical current passing through said electrical resistance, said electrical current monitor configured to record said a value representing measured electrical current at selected times, said electrical current monitor configured to report said recorded values representing measured electrical current and said selected times in response to an interrogation command.

The foregoing and other objects, aspects, features, and advantages of the invention will become more apparent from the following description and from the claims.

### BRIEF DESCRIPTION OF THE DRAWINGS

The objects and features of the invention can be better understood with reference to the drawings described below, and the claims. The drawings are not necessarily to scale, emphasis instead generally being placed upon illustrating the principles of the invention. In the drawings, like numerals are used to indicate like parts throughout the various views.

FIG. 1A is a graph of the microbial activity in methane-producing aquatic sediments from Puffer's Pond, Amherst, Mass. vs. the current density for several different samples, along with a linear least squares fit to the observed data.

FIG. 1B is a graph of the steady state currents and [2-¹⁴C]-acetate turnover rates in columns of methanogenic sediments. Error bars represent the standard deviation of the mean for the mineralization of [2-¹⁴C]-acetate in triplicate incubations of sediment subsampled from the depth that the currents were recorded.

FIG. 2A is a graph of the microbial activity (as determined by the rate that [2-¹⁴C]-acetate was converted to ¹⁴CH₄ and ¹⁴CO₂) vs. the current density for several different sulfate-reducing sediments from Nantucket Marine Station, Nantucket, Mass.

FIG. 2B is a graph of the steady state currents and [2-¹⁴C]-acetate turnover rates in columns of sulfate-reducing sediments. Error bars represent the standard deviation of the mean for the mineralization of [2-¹⁴C]-acetate in triplicate incubations of sediment subsampled from the depth that the currents were recorded.

FIG. 3A is a graph of the microbial activity (as determined by the rate that [2-¹⁴C]-acetate was converted to ¹⁴CH₄ and ¹⁴CO₂) vs. the current density for several different sediments from a subsurface site in Rifle, Colo. in which Fe(III) reduction was the terminal electron-accepting process.

FIG. 3B is a graph of the steady state currents and [2-¹⁴C]-acetate turnover rates in columns of Fe(III)-reducing sediments. Error bars represent the standard deviation of the mean for the mineralization of [2-¹⁴C]-acetate in triplicate incubations of sediment subsampled from the depth that the currents were recorded.

FIG. 4 is a diagram of the sediment incubation apparatus, operating according to principles of the invention.

FIG. 5 is a diagram that illustrates a model for current production with microbial activity sensors.

FIG. 6 is a schematic diagram of a current monitoring approach using sediment incubation cylinders.

FIG. 7 is an image of sediment incubations with current-monitoring using digital multimeters.

FIG. 8 is a schematic flow diagram of the operation of a microbial activity sensor at a remote (field) location.

### DETAILED DESCRIPTION

A simple strategy to estimate in situ microbial activity has been one of the holy grails in the fields of subsurface biogeochemistry and bioremediation. We recently discovered that microorganisms associated with graphite electrodes placed at depth within the subsurface can generate readily measurable currents and that the amount of current increases in response to an increase in acetate availability in the groundwater. Acetate is a central intermediate in the anaerobic degradation of organic matter, regardless of the terminal electron accepting process. These considerations suggested that it should be possible to estimate rates of microbial metabolism in a diversity of anaerobic subsurface environments from the current produced from electrodes embedded in the site of interest.

Our SMART (Subsurface Microbial Activity in Real Time) approach was evaluated in a diversity of soils and sediments in which either iron-reduction, sulfate, reduction, or methane production was the predominant terminal electron-accepting process. In all sediment types there was a strong direct correlation between current output from small electrodes emplaced in the soils/sediments and rates of anaerobic microbial metabolism as directly determined from the metabolism of [2-14C]-acetate to [14C]-carbon dioxide and [14C]-methane. SMART had a wide dynamic range, responding well at both high, intermediate, and low rates of natural organics degradation, as well as signaling sudden inputs of new organic sources. We also demonstrate a direct correlation between current production and rates of microbial activity as determined by the turnover of tracer [2-¹⁴C]-acetate in sediments in which Fe(III) reduction or sulfate reduction was the predominate terminal electron-accepting process.

These results suggest that the SMART strategy is a simple, inexpensive, and effective approach for real-time monitoring of rates of anaerobic microbial metabolism in the subsurface. SMART is applicable not only when organic electron donors are added to groundwater to promote anaerobic respiration, but also for monitoring microbial activity associated with natural attenuation of contaminants. Furthermore, the SMART strategy can also serve as a sensor to monitor the migration of contaminant plumes.

The present invention measures microbial activity associated with the degradation of the complex organic matter that is naturally present in soils and sediments.

Friedman et al. specifically poised their anodes to detect one type of microbial activity—the activity of microorganisms that can reduce ferric iron and humic substances. They did not recognize nor demonstrate that a wide range of microbial activities including sulfate reduction and methane production could also be reflected in current production. Our approach eliminates the need for poising electronics and estimates rates of a diversity of types of microbial activity.

The simplicity of our system permits long-term (years to decades) deployment.

An important feature in our invention is that we have documented with an independent method that there is a direct correlation between current levels and the rates of microbial activity. This was not done in any of the previous studies and is an important feature in interpreting the current output results. Furthermore Friedman et al. clearly stated that they did not have certainty that their currents were measuring microbial activity. In contrast, we have documented a direct correlation between microbial activity and current.

The invention is a simple and inexpensive method for real-time monitoring of the rates of microbial activity in anaerobic soils, sediments, and groundwater. The monitoring system comprises a graphite electrode (the anode) that is embedded in the anaerobic environment of interest with a connection with an insulated wire to a cathode. In preferred embodiments, the anode is in contact with soil, with sediment, or is immersed in the groundwater of a subsurface monitoring well. The cathode is placed on the soil surface or, in the case of aquatic sediments, in the water overlying the sediments. The cathode can be comprised of electrically conductive material such as graphite, fashioned in one of many geometries, such as a disk, rectangular stick, or a brush configuration with many fine graphite bristles. The electrical connection between the anode and the cathode contains a simple, inexpensive resistor. In proof-of-concept studies the resistor was 560 ohms, but other resistances are likely to be acceptable. The anode is colonized by microorganisms, native to the environment of interest, that are capable of oxidizing organic compounds, sulfur, and hydrogen with electron transfer to the anode. Microorganisms in the family Geobacteraceae are an example of bacteria that are capable of this process. Current between the anode and the cathode can be recorded with any commonly known device for measuring electric current.

Our results have demonstrated that there is a direct correlation between the current produced in these monitoring systems and the rates of microbial activity as verified by measurements of the rate that [2-¹⁴C] acetate is metabolized to [¹⁴C]-methane and [¹⁴C]-carbon dioxide in subsamples of the soils and sediments. This has been demonstrated with a range of sediment types in which either methane production, sulfate reduction, or ferric iron reduction was the predominant terminal electron-accepting process. The results were obtained with at a wide range of temperatures (4° C.-37° C.). These results show the broad applicability of the method.

Applications of the technology can include the measurement of microbial activities in soils and sediments for bioremediation where it is important to know whether microbially degradable pollutants are present and the rate at which they are being degraded, and in monitoring the flow of organic materials, such as plumes of materials emanating from various sources, such as spills, mining and drilling activities, and the like. Furthermore, there is a need for an instrument for scientists to measure microbial activity in soils and sediments.

We describe a simple non-poised anode system for monitoring the natural activity of a diversity of microorganisms. We demonstrate a direct correlation between current production and rates of microbial activity as determined by the turnover of tracer [2-¹⁴C]-acetate in sediments in which Fe(III) reduction, sulfate reduction, or methane production was the predominate terminal electron-accepting process.

### Methods

### Sediment Sources

Sediment was used from three separate sources. The first was Puffers Pond in Amherst, Mass. Puffers Pond has methanogenic sediment as evident from the bubbles that constantly surface in the pond. The second source is Nantucket marine sediment. The sediment is predominately sulfate reducing. The third source was an aquifer located in Rifle, Colo. Fe(III) reduction was the predominant electron-accepting process in these sediments. Sediments were added thoroughly mixed under anaerobic conditions and then added to core liners.

The core liners were constructed using 3 or 4 inch internal diameter PVC pipes. The internal diameter was varied according to the availability of sediment types. The PVC pipes were predrilled with holes so cores could be taken. The first set of holes was drilled three inches from the bottom of the pipe with two more sets drilled in 1.5 inch intervals up the tube. There were three or four holes drilled per set depending on the internal diameter of the tube; three holes for 3 inch ID and 4 for 4 inch ID. The bottom of the tube was sealed with either a PVC cap or rubber stopper and RTV sealant. Each of the holes was plugged with double or triple 0 rubber stoppers (Fisher Hampton, N.H.). Each cylinder was filled with 9 inches of sediment and an additional 9 inches of water was placed on top of the sediment. Cylinders were then placed in aquaria that were bubbled with N₂ or N₂:CO₂ (80:20) to guarantee an anaerobic environment for the sediment. Into each cylinder two anode/electrode pairs were added. The anode was a cylinder of graphite sealed in a 5 ml disposable pipet such that only a single 6.08 mm diameter section was at the end of the pipet. The graphite was epoxied with marine epoxy to marine grade wire while maintaining electrical contact between the graphite and the wire. The wire was connected to a 560Ω resistor. The anode assembly was then attached to a carbon bottle brush that acted as the cathode. The anode was inserted into the sediment such that the graphite face was aligned with either the set of holes 3 inches from the bottom or 6 inches from the bottom. The cathodes were placed such that the entirety of the brush was in the water above the sediment without touching the other cathode. Triplicate cylinders were placed in a temperature controlled environment.

The current density of the sensor was tracked until current production was at a steady state. Once current density reached a steady state for 4-10 days acetate turnover rates were determined.

### Acetate Turnover

Acetate turnover rate was determined with [2-C¹⁴]-acetate. Once current densities reached a steady state for 4-10 days sediments from the same depth as the exposed surface of the anode were sampled through the side ports with a 3 cm plastic syringe with the distal end cut off. Subcores were taken from the depths at which the electrodes were emplaced and the sediments extruded under anaerobic conditions into pre-weighed anaerobic 60 mL serum bottles that were then sealed with a thick butyl rubber stopper and weighed to determine core mass. The weight of the added sediment was determined and the sediments incubated in a water bath at the temperature at which the sediments had previously been incubated. A anaerobic solution (0.1 ml) of [2-¹⁴C]-acetate (available from American Radiolabeled Chemicals, Inc. St. Louis, Mo.; Specific Activity, 45 mCi/mmol; Purity, 99%) was injected into the sediments to provide 1.2-1.7 μCi. This added ca. 15 μM acetate to the sediment pore water.

Over time 0.5 ml of headspace was sampled with a syringe and needle and injected into a gas chromatograph (model GC-8A, available from Shimadzu, Kyoto, Japan) connected to a GC-RAM radioactivity detector (available from LabLogic Broomhill, UK) to determine the quantity of ¹⁴CH₄ and ¹⁴CO₂. The procedures described in Hayes L M, Nevin K P, Lovley D R. 1999. Role of prior exposure on anaerobic degradation of naphthalene and phenanthrene in marine harbor sediments. Organ. Geochem. 30:937-945) were used. The first order rate constants for acetate metabolism in each sample were calculated from the initial linear rate of ¹⁴CH₄ and ¹⁴CO₂ production according to k=f/t where f is the fraction of added label metabolized to product over an incubation time oft.

### Results

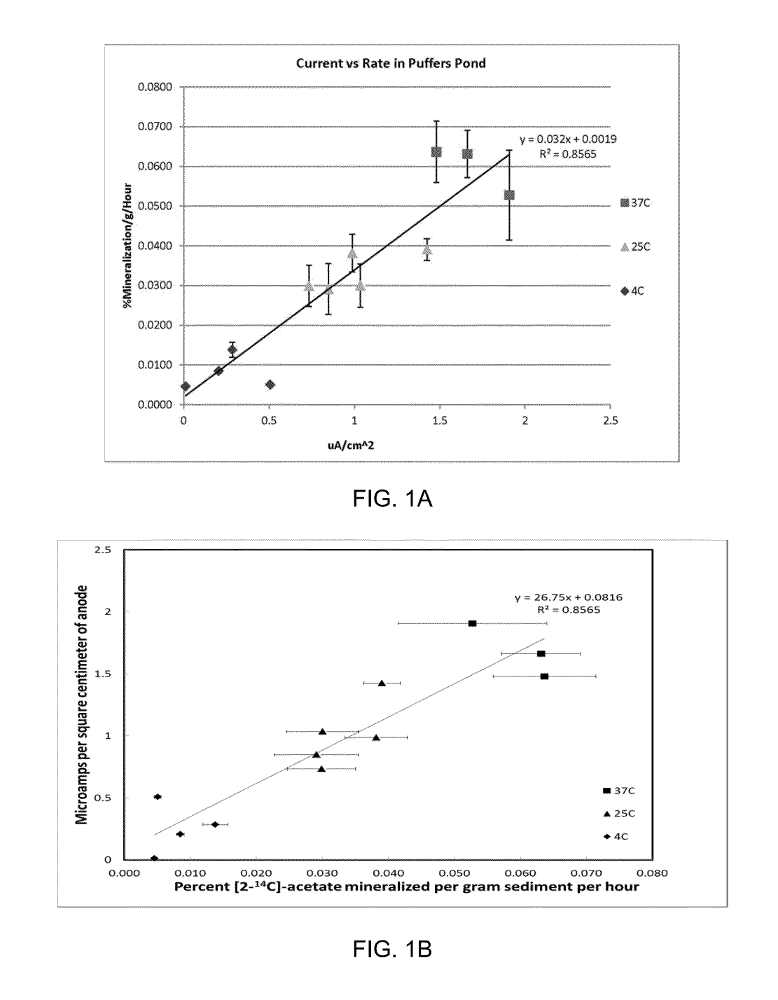

FIG. 1A is a graph of the microbial activity vs. the current density for several different freshwater methanogenic sediments from Puffer's Pond, Amherst, Mass.

Sediments were collected from Puffers Pond, Amherst, Mass. from areas where active methane gas ebullition was observed when a rod was inserted into the sediment. The water depth at sampling locations was 0.1 to 0.25 m. As described above for the Nantucket site sediments, the overlying oxidized sediment was removed and underlying sediment depth of approximately 5 to 25 cm was collected with a shovel into 20 liter plastic buckets, which were sealed with no headspace, and transported back to the laboratory. Sediments were stored at 15° C.

Incubating the sediments at different temperatures yielded different rates of microbial metabolism. There was a strong correlation between rates of metabolism as estimated from the rate of [2-¹⁴C]-acetate metabolism and current production rates.

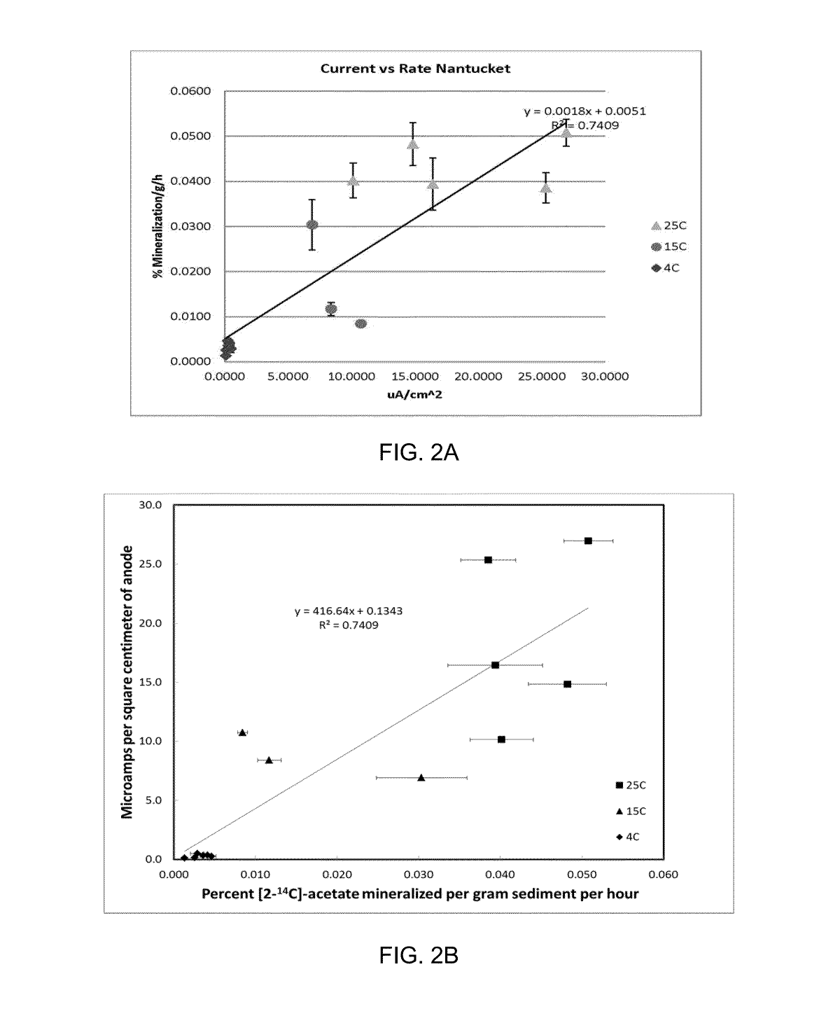

FIG. 2A is a graph of the microbial activity vs. the current density for several different sulfate-reducing sediments from Nantucket Marine Station, Nantucket, Mass. There was a positive correlation between current production and rates of [2-¹⁴C]-acetate metabolism at these sites as well. Sediments were collected from the site in Nantucket, Mass. as follows. At low tide, in the center of the salt marsh (water level 0.25 m), the oxidized zone (top 3-5 cm) was removed from the sediment in place and the underlying sediment depth of approximately 5 to 25 cm was collected by shovel, placed into mason jars, sealed without a headspace, and transported back to the laboratory. The sediments were stored at 15° C.

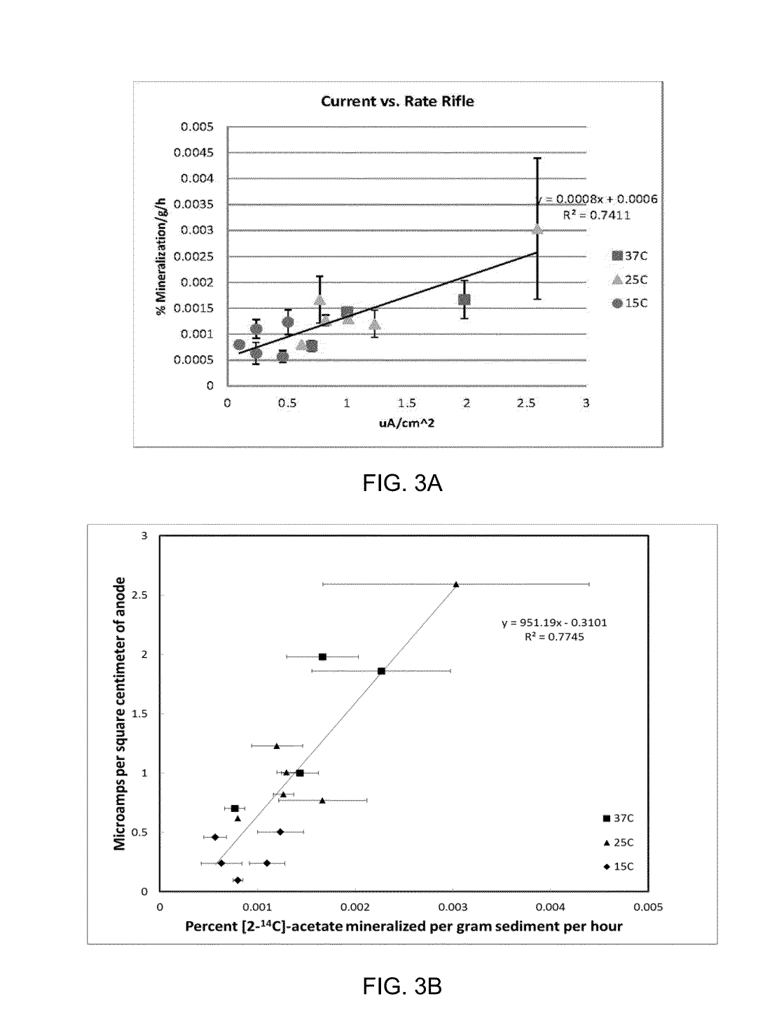

FIG. 3A and FIG. 3B are graphs of the microbial activity vs. the current density for several different sediments from a subsurface site in Rifle, Colo. in which Fe(III) reduction was the terminal electron-accepting process. Sediments were collected from a uranium-contaminated aquifer located in Rifle, Colo. Subsurface sediments were collected with a backhoe, stored in five gallon buckets, shipped to the laboratory at the University of Massachusetts, and stored at 15° C.

Sediment Incubations and Current Production

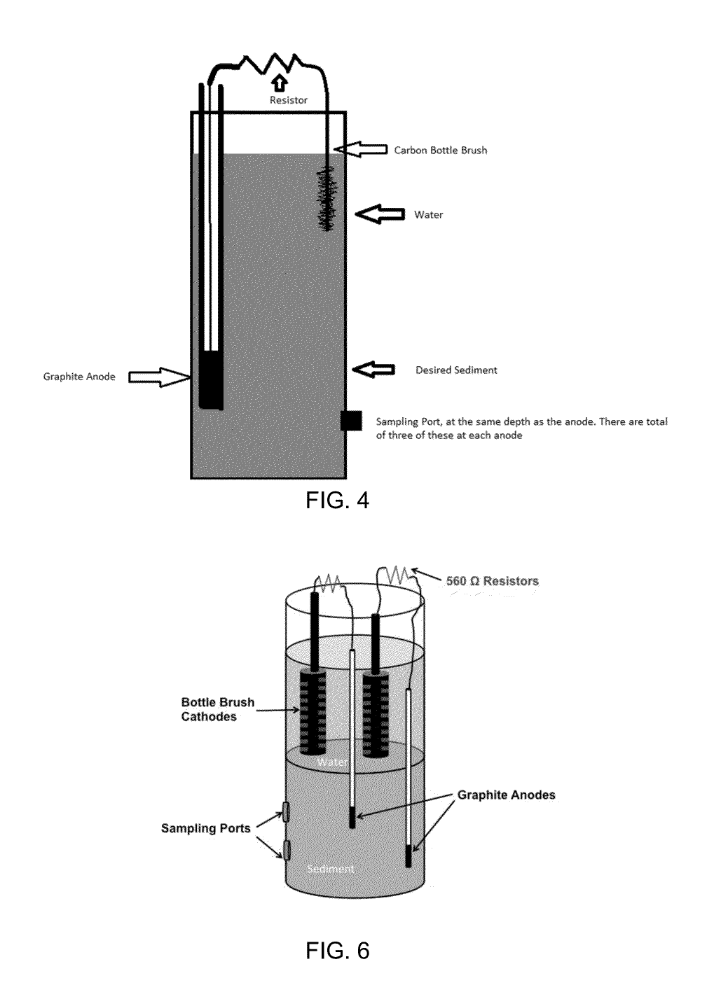

FIG. 4 is a diagram of the sediment incubation apparatus, operating according to principles of the invention.

The results demonstrate that there are strong correlations between the current output of a simple anode-resistor-cathode device and rates of anaerobic microbial activity in a diversity of soil/sediment types. This is the first example of monitoring the in situ microbial activity in soils and sediments with a simple system that does not employ a poised anode and the first study to directly compare current production rates with an independent estimate of the rates of microbial activity.

It is expected that this technology will have broad application in the real-time monitoring of microbial activity in a diversity of environments. It offers the possibility of continuous monitoring of microbial activity over time without disturbing the soils/sediments. The small size of the anodes and low cost of the materials makes it feasible to study heterogeneities in microbial activity at multiple scales both horizontally and vertically.

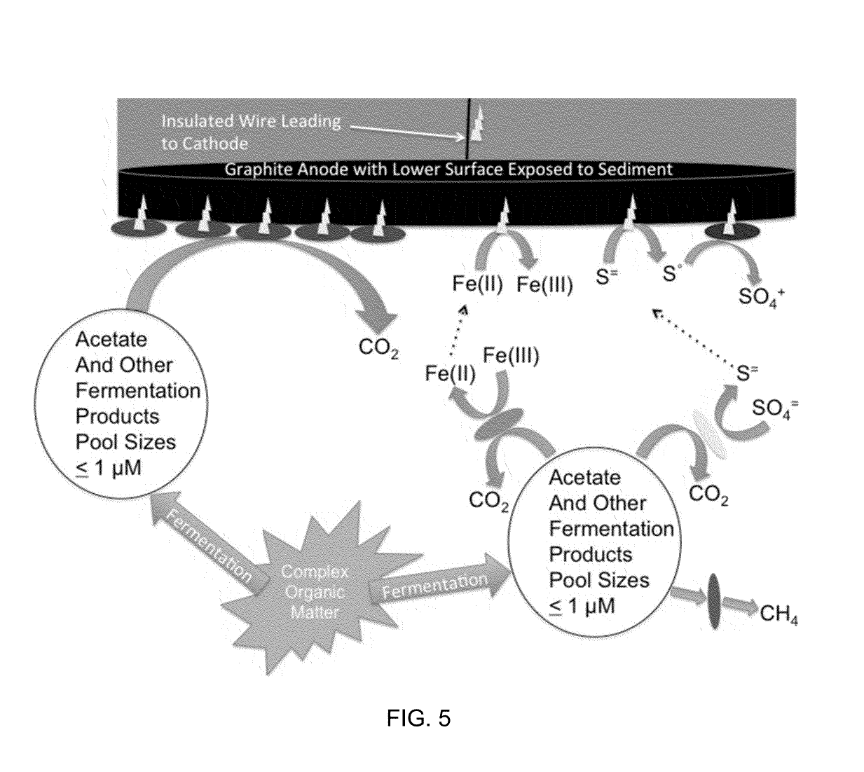

FIG. 5 is a diagram that illustrates a model for current production with microbial activity sensors. Acetate and other fermentation products produced from the hydrolysis and fermentation of particulate matter serve as electron donors for microbial current production at the anode surface. At distance from the anode these fermentation products are electron donors for methane production, sulfate reduction or Fe(III) reduction. Methane is not reactive with the anode, but Fe(II) and sulfide can be abiotically oxidized at the anode. Elemental sulfur produced from the oxidation of sulfide can serve as an electron donor for additional microbially catalyzed current production.

FIG. 6 is a schematic diagram of a current monitoring approach using sediment incubation cylinders.

Sediments were homogenized under a stream of N₂ in a 120 liter polyethylene container, fitted with a plastic top seal. The homogenized sediments were poured into PVC cylinders of either 7.6 cm diameter (Fe(III)-reducing sediments) or 10.2 cm diameter (sulfate-reducting or methanogenic sediments) that were sealed at the bottom with a butyl rubber stopper or PVC end caps (FIG. 6). The sediment height was 23 cm. Water from the respective sites was poured on top of the sediments to provide 23 cm of standing water above the sediment. There were holes (10.5 mm diameter) in the sides of the PVC cylinders, sealed with butyl rubber stoppers to provide ports for subsampling the sediments for [2-¹⁴C]-acetate turnover studies (FIG. 6).

The anodes were a graphite rod that sealed within a polystyrene pipet with marine epoxy such that just the end of the anode was exposed to the sediment, providing an accessible anode surface area of 28.26 mm² (FIG. 6). A marine-grade insulated wire was epoxied onto the anode and connected through a 560Ω resistor to a bottle brush carbon cathode (length, 12.3 cm; width, 2.7 cm). Two anode assemblies were inserted into each sediment column, either 8 or 16 cm from the bottom of the cylinder. The two cathodes were placed such that the entirety of the brush was in the water above the sediment without touching the other cathode. Triplicate cylinders were placed in temperature-controlled chambers with the cylinders submerged in water-filled aquaria. The sediments were incubated at a range of temperatures to provide a range of rates of microbial metabolism for each sediment type.

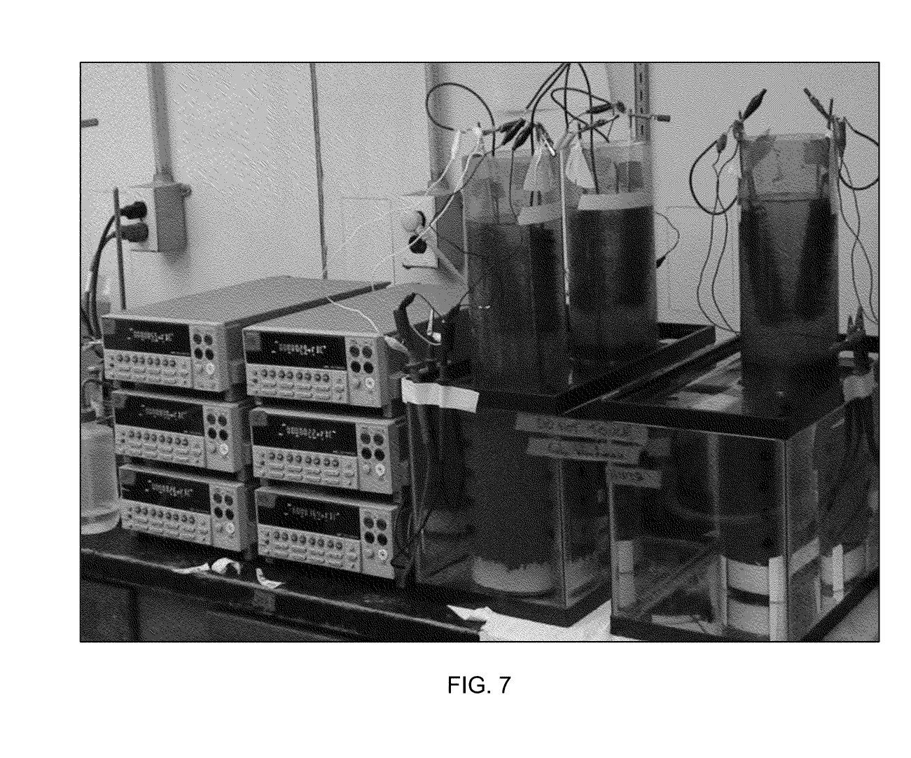

FIG. 7 is an image of sediment incubations with current-monitoring using digital multimeters. Current production in the methanogenic sediments was monitored with either a Keithley 2700 or 2000 Digital Multimeter (available from Keithley, Cleveland, Ohio) at hourly intervals. For the Fe(III)-reducing and sulfate-reducing sediments currents were monitored with a UEI DM284 Digital Multimeter (available from UEI, Beaverton, Oreg.) on a daily basis.

In order to determine whether the current produced at anodes emplaced in sediments could be correlated with rates of microbial metabolism at that location in the sediments, current production was compared with the rate of acetate mineralization. Acetate was chosen because it is the central intermediate in the anaerobic degradation of organic matter in sediments regardless of whether Fe(III) reduction, sulfate reduction, or methane production is the predominant terminal electron-accepting process. Therefore, rates of acetate metabolism in these types of anaerobic sediments are directly related to the overall rates that fermentable organic matter is being converted to carbon dioxide and methane.

It was hypothesized that current (I) would be directly related to the rate of acetate metabolism (Vₐ), according to:

I=Z×Vₐ (Reaction 1)

where Z is a correlation constant which is the sum of what may be a substantial number of complex factors controlling how much current is produced in the sediments. An understanding the many complex factors that may contribute to the Z term is not necessary in order to use current production as a proxy for microbial metabolism as long as Z is constant over the range of conditions evaluated (i.e. there is strong direct correlation between I and Vₐ).

Typically rates of acetate metabolism (Vₐ) are estimated from the first order rate constant of the metabolism of radiolabelled acetate (k) and the concentration of acetate (A) where

Vₐ=k×A (Reaction 2).

However, acetate concentrations in all three sediment types were below our detection limit of 10 μM with high performance liquid chromatography, preventing calculation of Vₐ. This added another unknown and combining reactions 1 and 2:

I=Z×k×A (Reaction 3).

At steady state, acetate concentrations acetate concentrations are controlled by the affinity of the microorganisms consuming the acetate and thus acetate concentrations are expected to be similar in sediments in which the same terminal electron-accepting predominates. Therefore, within sediments with the same terminal electron-accepting process A can be considered a constant and, if the hypothesis of a direct correlation between current production and acetate metabolism holds, then there will be a direct correlation between current and the first order rate constant for acetate metabolism with the product of the two constants Z and A as the correlation coefficient:

I=(ZA)×k (Reaction 4).

In fact, there was a direct correlation between the first order rate constant for acetate metabolism and current produced in all three sediment types investigated (FIG. 1B, FIG. 2B, FIG. 3B). As expected, the rate constant for acetate metabolism in the subsurface sediments from the Rifle, Colo. site were much lower than for the freshwater or marine surface sediments, reflecting the higher organic content of the two surface sediments. With all sediments, incubation at different temperatures was an effective method for providing a range of different rates of microbial metabolism in each sediment type.

Although the acetate rate constants in the freshwater sediments in which methane production predominated and the marine sediments in which sulfate reduction predominated were similar, the currents produced in the marine sediments for comparable acetate turnover times were ca. 15-fold higher (FIG. 1B, FIG. 2B), suggesting that the factor ZA was ca. 15-fold larger in the sulfate-reducing sediments. The higher ZA term for the sulfate-reducing sediments cannot be attributed to higher acetate concentrations. Sulfate reducers have a higher affinity for acetate than methanogens, thus the acetate pool is expected to be lower in sediments in which sulfate reduction predominates. In fact acetate measurements in sediments similar to those studied here revealed that the acetate pool in methanogenic sediments was twice as high as in sulfate-reducing sediments. This suggests that one or more of the many factors contributing to Z was greater in the sediments in which sulfate reduction was the terminal electron-accepting process.

One possibility is that there was an additional source of electron donor for current production in the sulfate-reducing sediments that was not available in the methanogenic sediments. In both sediment types, the production of acetate, as well as H₂ and minor fermentation acids, near the anode surface is expected to supply electron donors for current production (FIG. 5). At distance from the anode these electron donors support the reduction of sulfate or the production of methane. Methane is highly unreactive and is not likely to abiotically interact with the anode or to serve as an electron donor for microbially catalyzed current production. However, sulfide produced from sulfate reduction is highly reactive and is abiotically oxidized to elemental sulfur at anode surfaces (31, 32). A diversity of microbes (20, 21) can oxidize the elemental sulfur to sulfate with further current production (FIG. 5). Therefore, microbial metabolism at greater distances from the anode can be captured as current production in marine sediments than is possible in methanogenic sediments.

These considerations suggest that although there is a direct correlation between current production and microbial activity in sediments in which methane production or sulfate reduction is the predominant terminal electron-accepting process, a different calibration will be needed to infer rates of microbial activity from specific current levels in the two types of sediments. Therefore, measurements of dissolved H₂, or some other technique to determine the predominant terminal electron-accepting process will be important when interpreting current outputs to monitor microbial activity in environments in which there can be shifts between sulfate reduction and methane production.

In the Fe(III)-reducing sediments currents were more comparable to those in the sulfate-reducing sediments at similar acetate-turnover rates, and much higher than in the methanogenic sediments. As in the sulfate-reducing environments, microbial activity at distance from the anode in Fe(III)-reducing sediments may be reflected in current production at the anode because Fe(II) produced from Fe(III) reduction can diffuse to the anode and donate electrons (FIG. 5).

The results demonstrate that there are strong correlations between the current output of a simple anode-resistor-cathode device and rates of anaerobic microbial activity in a diversity of anaerobic sediments. This is the first example of monitoring the in situ microbial activity in soils and sediments with a simple system that does not employ a poised anode and the first study to directly compare current production rates with an independent estimate of the rates of microbial activity.

It is expected that this technology will have broad application in the real-time monitoring of anaerobic microbial activity in a diversity of submerged soils as well as sediments. It offers the possibility of continuous monitoring of microbial activity over time without disturbing the soils/sediments. The small size of the anodes and low cost of the materials makes it feasible to study heterogeneities in microbial activity at multiple scales both horizontally and vertically. At the present stage of development, this SMART (Subsurface Microbial Activity in Real Time) technology will primarily be useful for monitoring relative changes in microbial activity in response to environmental perturbations, such as the response to temperature change shown here. However, other applications, such as deploying electrodes at the periphery of polluted sites as a sentinel to detect the migration of organic contaminants, are under investigation.

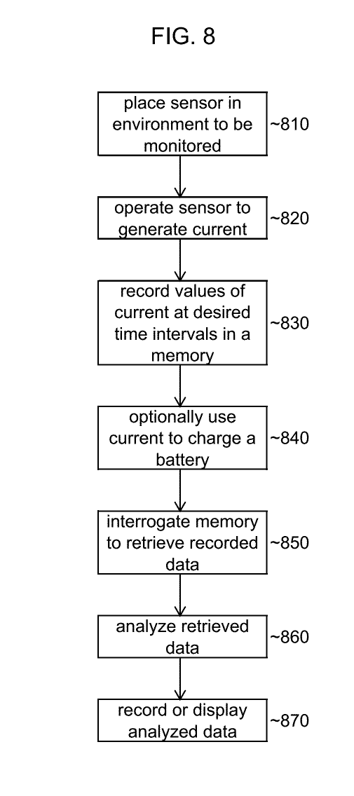

FIG. 8 is a schematic flow diagram of the operation of a microbial activity sensor at a remote (field) location. In step 810 one places a sensor constructed according to the principles of the invention in an environment to be monitored. In step 820 one causes the sensor to operate to generate current. In step 830 one records the values of current at desired time intervals in a memory. The recorded data includes both a value for the current and a respective time when that value was observed or recorded. In step 840 one can optionally use the current to charge a battery (e.g., a secondary battery, that is, one that can be recharged). The battery can be used to power electronics used for measurement, and/or for receiving and transmitting recorded data or other information. Alternatively, one can power the device with other power sources, such as photovoltaic solar cells, or a primary battery (e.g., one that cannot be recharged). In step 850 one can interrogate the memory to retrieve the recorded data. In step 860 one analyzes the retrieved data. In step 870 the analyzed data is recorded or displayed to a user.

Monitoring in situ microbial activity in anaerobic submerged soils and aquatic sediments can be labor intensive and technically difficult, especially in dynamic environments in which a record of changes in microbial activity over time is desired. Microbial fuel cell concepts have previously been adapted to detect changes in the availability of relatively high concentrations of organic compounds in waste water but, in most soils and sediments, rates of microbial activity are not linked to the concentrations of labile substrates, but rather to the turnover rates of the substrate pools with steady state concentrations in the nM-μM range. In order to determine whether levels of current produced at a graphite anode would correspond to the rates of microbial metabolism in anaerobic sediments, small graphite anodes were inserted in sediment cores and connected to graphite brush cathodes in the overlying water. Currents produced were compared with the rates of [2-¹⁴C]-acetate metabolism. There was a direct correlation between current production and the rate that [2-¹⁴C]-acetate was metabolized to ¹⁴CO₂ and ¹⁴CH₄ in sediments in which Fe(III) reduction, sulfate reduction, or methane production was the predominant terminal electron-accepting process. At comparable acetate turnover rates, currents were higher in the sediments in which sulfate-reduction or Fe(III) reduction predominated than in methanogenic sediments. This was attributed to reduced products (Fe(II), sulfide) produced at distance from the anode contributing to current production in addition to the current that was produced from microbial oxidation of organic substrates with electron transfer to the anode surface in all three sediment types. The results demonstrate that inexpensive graphite electrodes may provide a simple strategy for real-time monitoring of microbial activity in a diversity of anaerobic soils and sediments.

### DEFINITIONS

Unless otherwise explicitly recited herein, any reference to an electronic signal or an electromagnetic signal (or their equivalents) is to be understood as referring to a non-transitory electronic signal or a non-transitory electromagnetic signal.

Recording the results from an operation or data acquisition, such as for example, recording results at a particular frequency or wavelength, is understood to mean and is defined herein as writing output data in a non-transitory manner to a storage element, to a machine-readable storage medium, or to a storage device. Non-transitory machine-readable storage media that can be used in the invention include electronic, magnetic and/or optical storage media, such as magnetic floppy disks and hard disks; a DVD drive, a CD drive that in some embodiments can employ DVD disks, any of CD-ROM disks (i.e., read-only optical storage disks), CD-R disks (i.e., write-once, read-many optical storage disks), and CD-RW disks (i.e., rewriteable optical storage disks); and electronic storage media, such as RAM, ROM, EPROM, Compact Flash cards, PCMCIA cards, or alternatively SD or SDIO memory; and the electronic components (e.g., floppy disk drive, DVD drive, CD/CD-R/CD-RW drive, or Compact Flash/PCMCIA/SD adapter) that accommodate and read from and/or write to the storage media. Unless otherwise explicitly recited, any reference herein to “record” or “recording” is understood to refer to a non-transitory record or a non-transitory recording.

As is known to those of skill in the machine-readable storage media arts, new media and formats for data storage are continually being devised, and any convenient, commercially available storage medium and corresponding read/write device that may become available in the future is likely to be appropriate for use, especially if it provides any of a greater storage capacity, a higher access speed, a smaller size, and a lower cost per bit of stored information. Well known older machine-readable media are also available for use under certain conditions, such as punched paper tape or cards, magnetic recording on tape or wire, optical or magnetic reading of printed characters (e.g., OCR and magnetically encoded symbols) and machine-readable symbols such as one and two dimensional bar codes. Recording image data for later use (e.g., writing an image to memory or to digital memory) can be performed to enable the use of the recorded information as output, as data for display to a user, or as data to be made available for later use. Such digital memory elements or chips can be standalone memory devices, or can be incorporated within a device of interest. “Writing output data” or “writing an image to memory” is defined herein as including writing transformed data to registers within a microcomputer.

“Microcomputer” is defined herein as synonymous with microprocessor, microcontroller, and digital signal processor (“DSP”). It is understood that memory used by the microcomputer, including for example instructions for data processing coded as “firmware” can reside in memory physically inside of a microcomputer chip or in memory external to the microcomputer or in a combination of internal and external memory. Similarly, analog signals can be digitized by a standalone analog to digital converter (“ADC”) or one or more ADCs or multiplexed ADC channels can reside within a microcomputer package. It is also understood that field programmable array (“FPGA”) chips or application specific integrated circuits (“ASIC”) chips can perform microcomputer functions, either in hardware logic, software emulation of a microcomputer, or by a combination of the two. Apparatus having any of the inventive features described herein can operate entirely on one microcomputer or can include more than one microcomputer.

General purpose programmable computers useful for controlling instrumentation, recording signals and analyzing signals or data according to the present description can be any of a personal computer (PC), a microprocessor based computer, a portable computer, or other type of processing device. The general purpose programmable computer typically comprises a central processing unit, a storage or memory unit that can record and read information and programs using machine-readable storage media, a communication terminal such as a wired communication device or a wireless communication device, an output device such as a display terminal, and an input device such as a keyboard. The display terminal can be a touch screen display, in which case it can function as both a display device and an input device. Different and/or additional input devices can be present such as a pointing device, such as a mouse or a joystick, and different or additional output devices can be present such as an enunciator, for example a speaker, a second display, or a printer. The computer can run any one of a variety of operating systems, such as for example, any one of several versions of Windows, or of MacOS, or of UNIX, or of Linux. Computational results obtained in the operation of the general purpose computer can be stored for later use, and/or can be displayed to a user. At the very least, each microprocessor-based general purpose computer has registers that store the results of each computational step within the microprocessor, which results are then commonly stored in cache memory for later use, so that the result can be displayed, recorded to a non-volatile memory, or used in further data processing or analysis.

### Theoretical Discussion

Although the theoretical description given herein is thought to be correct, the operation of the devices described and claimed herein does not depend upon the accuracy or validity of the theoretical description. That is, later theoretical developments that may explain the observed results on a basis different from the theory presented herein will not detract from the inventions described herein.

Any patent, patent application, patent application publication, journal article, book, published paper, or other publicly available material identified in the specification is hereby incorporated by reference herein in its entirety. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

While the present invention has been particularly shown and described with reference to the preferred mode as illustrated in the drawing, it will be understood by one skilled in the art that various changes in detail may be affected therein without departing from the spirit and scope of the invention as defined by the claims.

What is claimed is:

1. A microbial activity sensor, comprising:
an anode electrode configured to support a biofilm on a surface thereof, and configured to be imbedded in the ground, in sediment, or immersed in groundwater, said anode electrode having an anode electrode terminal;
a cathode electrode in electrical contact with the ground, sediment, or groundwater, said cathode electrode having a cathode electrode terminal;
an electrical resistance connected between said anode electrode terminal and said cathode electrode terminal; and
an electrical current monitor in electrical communication with said electrical resistance, said electrical current monitor configured to measure an electrical current passing through said electrical resistance, said electrical current monitor configured to record said a value representing measured electrical current at selected times, said electrical current monitor configured to report said recorded values representing measured electrical current and said selected times in response to an interrogation command.

2. The microbial activity sensor of claim 1, further comprising a communication device configured to receive said interrogation command, and configured to provide said recorded values representing measured electrical current and said selected times in response to said interrogation command.

3. A microbial activity monitoring method, comprising the steps of:
providing an microbial activity sensor, comprising:
an anode electrode configured to support a biofilm on a surface thereof, and configured to be imbedded in the ground, in sediment, or immersed in groundwater, said anode electrode having an anode electrode terminal;
a cathode electrode configured to be in electrical contact with the ground, sediment, or groundwater, said cathode electrode having a cathode electrode terminal;
an electrical resistance connected between said anode electrode terminal and said cathode electrode terminal; and
an electrical current monitor in electrical communication with said electrical resistance, said electrical current monitor configured to measure an electrical current passing through said electrical resistance, said electrical current monitor configured to record said a value representing measured electrical current at selected times, said electrical current monitor configured to report said recorded values representing measured electrical current and said selected times in response to an interrogation command;
operating said microbial activity sensor to generate values representing measured electrical current at selected times;
recording said values representing measured electrical current and respective selected times;
interrogating said microbial activity sensor to recover said values representing measured electrical current and respective selected times;
analyzing said values representing measured electrical current and respective selected times to produce a result representing a level of microbial activity; and
performing at least one of recording said result, transmitting said result to a data handling system, or to displaying said result to a user.
